(19) United States
(12) Patent Application Publication
(10) Pub. No.: US 2005/0288603 A1
(43) Pub. Date: Dec. 29, 2005

(54) Method for obtaining and displaying urethral pressure profiles

(76) Inventor: Ing Han Frank Goping, Oakville (CA)

Correspondence Address: CAHN & SAMUELS LLP, 2000 P STREET NW, SUITE 200, WASHINGTON, DC, 20036, US

(21) Appl. No.: 10/874,582

(22) Filed: Jun. 24, 2004

(51) Int. Cl. A61B 5/00

(52) U.S. Cl. 600/561

(57) ABSTRACT

A method for performing urodynamic testing on mammals. A pressure sensor, adapted to transmit the pressure within the urethra to a data monitoring device, is placed a first position within a urethra of the mammal. The urethral pressure is measured while the mammal undergoes at least one stress maneuver. A first, maximal and intermediate urethral pressure is measured during the stress maneuver. The pressure sensor is then moved to a second position within the urethra and the steps of performing the stress maneuver, selecting a stress maneuver and selecting the first, maximal and intermediate urethral pressures are performed at this second position. After completion of these steps a timewise representation of urethral pressures at the first position and the second position during the pressure events is displayed on a display derived from the first, maximal and intermediate urethral pressures.

### FIELD OF THE INVENTION

This invention relates to the field of urodynamics and more specifically to methods for obtaining and displaying urethral pressure profiles.

### BACKGROUND OF THE INVENTION

The urethra is a tube in mammals that carries urine from the bladder out of the body. The urethra includes a urinary sphincter to prevent the release of urine from the bladder until urination occurs. When the time comes for urination, the bladder contracts, the sphincter is opened and the urine within the bladder is released.

However, there are a wide variety of situations in which the control over urination is not maintained. A dysfunction of the urinary sphincter may result in incontinence. If the urinary sphincter is not applying sufficient force to counteract the fluid pressure within the bladder, leakage of urine may occur.

Urethral pressure profiles have been used since the 1970's to measure pressures within the urethra. The pressure profiles have been used to assist clinicians with determining the causes of incontinence and other urinary problems. The urethral pressure profile procedure involves placing a urethral catheter within the urethra towards the bladder. The catheter includes a pressure sensor which is connected to a data monitoring device (e.g. a computer, a data plotter, etc.) The clinician withdraws the catheter using a motor operating at a constant speed. The pressure is monitored on a continuous basis (in the case of an analog data monitor by a data plotter) or at a particular sampling rate (in the case of a digital data monitor). Often, the clinician would ask the patient to cough during the procedure at various intervals. The resulting pressure data is plotted as a function of urethral distance.

However, digital data monitors to date have suffered from low sampling rates. As a result, with a transient event such as a cough, only a few data points were measured and the clinician could not know if one of those points was the peak pressure. In addition, as it is rare that a patient will cough to the same intensity every time, the test may show a low pressure point along the urethra when, in fact, the patient simply did not cough as hard. In such a case, a misdiagnosis may occur. To overcome these drawbacks, the test may need to be performed a few times, resulting in significant discomfort for the patient.

In addition, the urethral pressure profiles are a relatively crude manner to display complex urethral stress events, such as a cough. The clinician cannot readily view the manner in which the stress events affect the urethra.

Therefore, an improved method for performing urethral pressure test and for viewing the results is needed.

### SUMMARY

One aspect of this invention is a method for performing urodynamic testing on mammals. The first step of this method involves inserting a pressure sensor to a first position within a urethra of the mammal. The pressure sensor is adapted to transmit the pressure within the urethra to a data monitoring device. The pressure within the urethra of the mammal is then measured while the mammal undergoes at least one stress maneuver. One of the at least one stress maneuvers is selected as an accepted stress maneuver. A first urethral pressure is measured prior to the accepted stress maneuver. A maximal urethral pressure measured during the accepted stress maneuver. An intermediate pressure measured between the first urethral pressure and the maximal urethral pressure, the intermediate pressure occurring at a time interval from the start of stress maneuver. The pressure sensor is then moved to a second position within the urethra and the steps of performing the stress maneuver, selecting a stress maneuver and selecting the first, maximal and intermediate urethral pressures are performed at this second position.

After completion of these steps a timewise representation of urethral pressures at the first position and the second position during the pressure events is displayed on a display derived from the first, maximal and intermediate urethral pressures.

Optionally, the pressure sensor may include a fluid-filled element (such as a balloon), an electronic microtip, or an open-perfused microtip.

In an alternative embodiment to the present invention, the timewise representation of urethral pressures may be a series of graphs displaying urethral pressure as a function of urethral location. One of the graphs may display the first urethral pressure at the first position and the second position. Another of the graphs may display the maximal urethral pressure at the first position and the second position. Another of the graphs may display the intermediate urethral pressure at the first position and the second position.

In further alternative, the steps of performing the stress maneuver, selecting a stress maneuver and selecting the first, maximal and intermediate urethral pressures are performed at a third position position. A timewise representation of urethral pressures is a series of graphs displaying urethral pressure as a function of urethral location. One of the graphs displays a pressure at the first position, the second position and the third position selected from the group consisting of: the first urethral pressure, the maximal urethral pressure and the intermediate pressure. The pressures may be displayed as points on the graph. The points may be joined using a curve-fitting algorithm.

The pressure in the bladder of the mammal may also be measured.

Optionally, the stress maneuver may be a cough or a Valsalva maneuver performed by the mammal.

In yet a further alternative, the pressure sensor may be moved within the urethra using a motorized puller.

In still a further alternative, the mammal is observed for indications of urinary leakage.

In another alternative, the pressure in the bladder of the mammal is also measured and each of the measured urethral pressures is expressed as a percentage of bladder pressure.

Optionally, stress profiles for each of the first position and the second position is prepared. Each of the stress profile displays the first urethral pressure, the maximal urethral pressure and the intermediate urethral pressure as a function of time. The stress profiles may be normalized with respect to the bladder pressure.

In a further option, the maximal urethral pressure is selected by selecting the pressure measured at a preselected time interval from the start of the stress maneuver. Similarly, the intermediate urethral pressure may be selected by selecting the pressure measured at another preselected time interval from the start of the stress maneuver.

The first position and the second position may be selected at predetermined distances from the opening of the urethra.

In another aspect of the present invention, a method for displaying urethral pressure profiles for mammals is described. The first step of the method is obtaining pressure measurements at a plurality of locations within the urethra of a mammal while the mammal undergoes a stress maneuver. The pressure measurements are plottable on a pressure-time graph as a stress profile. A first pressure measurement is selected from each location within the urethra used in the first step. The first pressure measurements are selected such that they occur at substantially corresponding points in the respective stress profiles. The first pressure measurements form a first set of profile pressures. Similarly, a second pressure measurement is selected from those measurements in the first step from each location within the urethra used in the first step. The second pressure measurements are selected such that they occur at substantially corresponding points in the respective stress profiles. The second pressure measurements form a second set of profile pressures.

Each of the first pressure measurements is plotted on a first graph of pressure as a function of urethral location. Similarly, each of the second pressure measurements is plotted on a second graph of pressure as a function of urethral locations. Each of the graphs is then displayed in sequential manner.

Optionally, the first pressure measurements are joined together on a curve. The curve may be calculated using a curve-fitting algorithm.

In another embodiment, the stress profiles may normalized with respect to a preselected pressure.

In yet another embodiment, the graphs may be displayed on a computer display.

### BRIEF DESCRIPTION OF THE DRAWINGS

The novel features which are believed to be characteristic of the present invention, as to its structure, organization, use and method of operation, together with further objectives and advantages thereof, will be better understood from the following drawings in which presently preferred embodiment(s) of the invention will now be illustrated by way of example. It is expressly understood, however, that the drawings are for the purpose of illustration and description only and are not intended as a definition of the limits of the invention. Embodiments of this invention will now be described by way of example in association with the accompanying drawings in which:

FIG. 1 is a schematic of a system for conducting urethral pressure profile testing;

FIG. 2 is a typical urethral pressure profile in accordance with the prior art;

FIG. 3 is a graph showing urethral pressure measurements as a function of time during a standard stress maneuver;

FIG. 4 is a graph showing urethral pressure measurements as a function of time during a weak stress maneuver;

FIGS. 5A through 5D are a series of graphs showing urethral pressure measurements as a function of time during a standard abdominal stress event at different positions; and

FIGS. 6A through 61 are a series of graphs showing urethral pressure profiles at different times during an abdominal stress event.

### DESCRIPTION OF THE PREFERRED EMBODIMENTS

The novel features which are believed to be characteristic of the present invention, as to its structure, organization, use and method of operation, together with further objectives and advantages thereof, will be better understood from the following discussion in combination with the accompanying drawings.

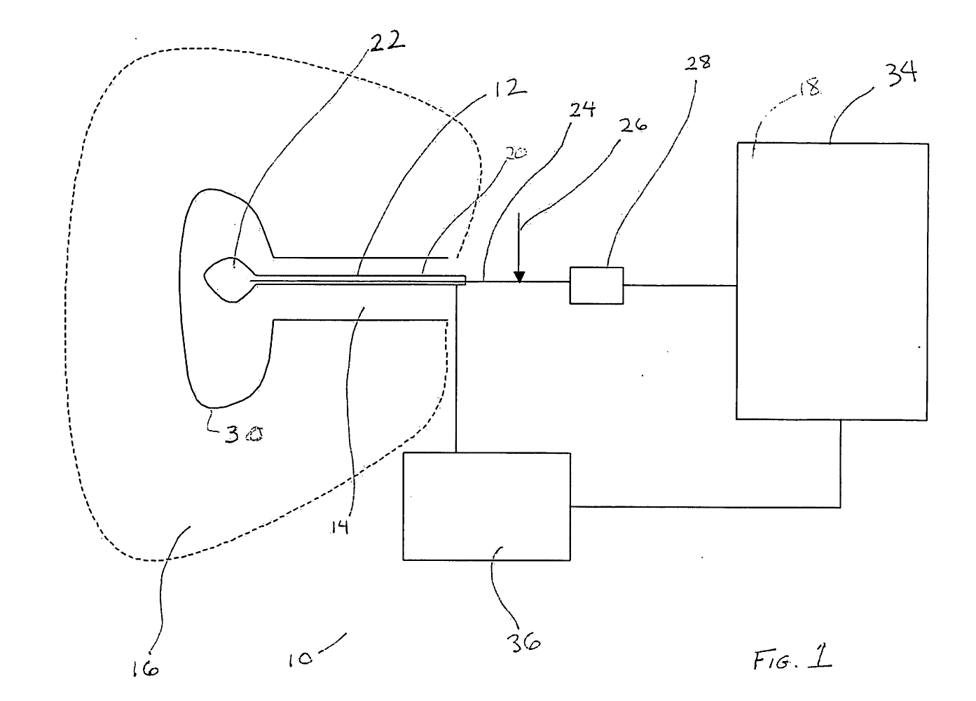

FIG. 1 is a schematic of a typical urodynamic testing system 10 in accordance with the present invention. System 10 includes a pressure sensor 12 placed inside the urethra 14 of a patient 16. Pressure sensor 12 is connected to a data monitoring device 18 which records the pressures at given locations in the urethra.

Pressure sensor 12 preferably comprises a catheter 20 having a fluid filled balloon 22 at the tip thereof. Catheter 20 preferably contains at least one lumen 24 in fluid communication with balloon 22. Catheter 20 may also include an inlet port 26 and a pressure transducer 28. A fluid (such as a gas or saline) may be used to fill the balloon 22 and the lumen 24 to a known first pressure. Inlet port 26 is preferably sealed after filling the balloon 22 and the lumen 24 so that a fixed pressure of the fluid is maintained therein. Pressure transducer 28 is operatively connected to the fluid within lumen 24 to obtain the pressure of the fluid therein. Pressure transducer 28 passes the pressure measurement to data monitoring device 18 for data storage.

A variety of pressure sensors known in the art may be used instead of the catheter 20 described above. For example the pressure sensor can be and open electronic microchip, an air or water filled balloon membrane as discussed above, or a fluid-perfused open hole catheter which allows for constant out-flowing of fluid or gas. Optionally, catheter 20 may include a second lumen for filling the patient's bladder 30 with water or other fluids. Catheter 20 may include two or more balloons at different positions to measure the pressures simultaneously at different positions in the urethra.

Preferably, where pressure sensor 12 includes a catheter 20, catheter 20 includes a plurality of measurement markings so that the clinician performing the test can determine the length of catheter within the patient.

Data monitoring device 18 is preferably a computer 34 having software running thereon for recording the pressures obtained by pressure transducer 28. Computer 34 may be a personal computer, mainframe, personal digital assistant, dedicated terminal or other data recording device. Alternatively, data monitoring device 18 may include an analog printer or plotter, although data would then need to be manually transferred to another computing device for processing.

A typical urethral pressure profile, as shown in FIG. 2, may be obtained using the equipment as described above. For a typical test, the clinician will insert catheter 20 into the urethra 14 of a patient 16. The clinician will infuse the balloon 22 and the lumen 24 with a known volume of fluid via inlet port 26. The data monitoring device 18 is activated to record the pressure on the balloon 22 as determined using pressure transducer 28. A motor 36 is affixed to the catheter 20 and pulls it out of the urethra at a predetermined rate. The data monitoring device records 18 records and stores the pressure at fixed sampling intervals while catheter 20 is removed. In some cases, the patients may be asked to have one or more stress maneuver in the nature of an abdominal stressor event (e.g. a cough) while the catheter is removed. After the catheter is removed, the data monitoring device 18 provides a graph of urethral wall pressure as a function of urethral length. In actuality, the graph is one of urethral wall pressure as a function of time, but the time is converted to length using the motor speed. The clinician can then view the urethral pressure profile so obtained to assist in the diagnosis of the patient.

The method of the present invention varies significantly from the method described above. In the present method, the clinician seeks to obtain a plurality of measurements during stress maneuvers at a number of locations along the urethra. Preferably, relatively high sampling rates (typically between 10 and 100 Hz, preferably between 20 and 50 Hz) are used for transferring pressure measurements from the transducer to the data monitoring device.

Using this method, the data monitoring device 18 is operatively connected to motor 36 to control its operation. The clinician selects the number of measurements to be made and the location of those measurements. For example, if the patient has a typical urethral length of 5 cm (in the case of a female patient), the clinician may wish to measure the urethral pressures during coughs at four different points (e.g. 4 cm, 3 cm, 2 cm and 1 cm from the urethral opening). The clinician enters the number of points and the location of the points into the data monitoring device or, alternatively, the clinician may be presented with a preset template where this information is preset.

The clinician will then place the catheter 20 within the urethra 14 of the patient and record the length of the catheter 20 placed within the urethra 14 as indicated by measurement markings. This measurement will typically be recorded in the data monitoring device 18.

The data monitoring device 18 will then activate the motor 36 to remove the catheter from the patient at a fixed rate for a fixed time interval until the first measurement point is reached. The data monitoring device 18 will then stop the motor 36 from pulling the catheter 20 any further and continue to measure the pressure readings.

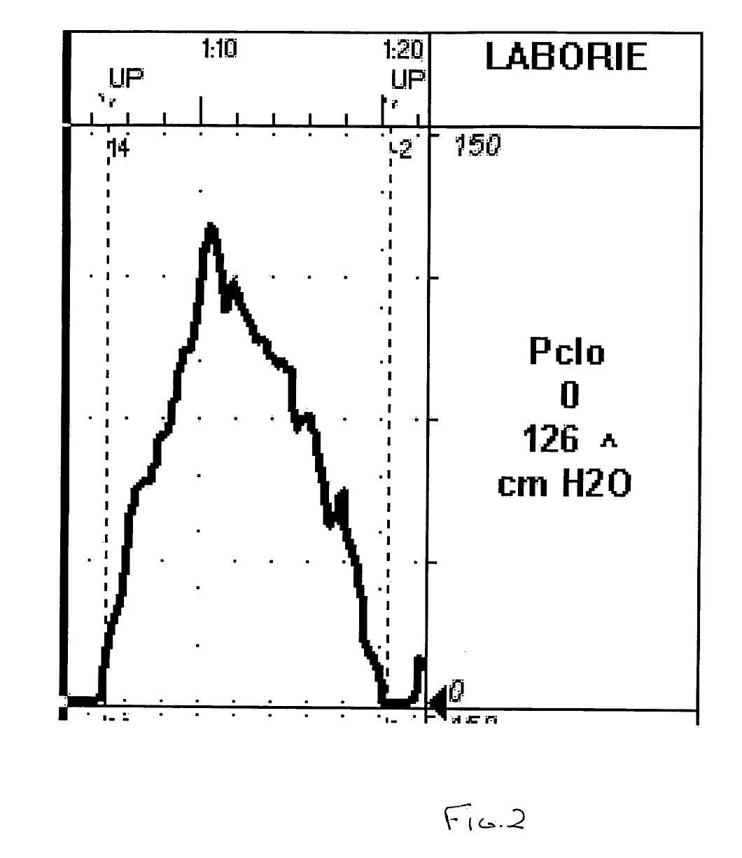

At this stage, the clinician will instruct the patient to cough. A cough will typically cause the muscle surrounding the urethra to compress the urethral walls about the balloon 22, increasing the pressure within the balloon 22 and the lumen 24 over a half second time period. FIG. 3 shows a typical urethral pressure-time graph for a cough. The pressure-time graph is displayed on the data monitoring device 18. As any one cough may be different from another, even in the same patient, the clinician will review the pressure-time graph to determine if the patient used sufficient force for the purposes of the test. (For the purposes of this description, this type of pressure-time graph will be referred to as a ‘stress profile’ or ‘cough profile’.) If the clinician determines that a more forceful cough is required (a weak cough profile is shown in FIG. 4), the patient may be asked to cough again after the clinician has adjusted the data monitoring device 18 to accept a new set of readings. If the clinician determines that the cough was sufficiently forceful, the clinician will instruct the data monitoring device 18 to continue with the test. Optionally, the clinician can also note whether there was leakage of urine from the urethra during the valid cough.

In one alternative to the method above, the clinician has the patient perform multiple coughs of varying intensity. The clinician can correlate coughs of similar intensities at various urethral locations.

Alternatively, the first measurement point reading may constitute a baseline against which further stressor measurements are compared for sufficiency of coughing force. In a further alternative, the data monitoring device 18 may determine the peak pressure (data point 80 on FIG. 3) and compare it to a predetermined pressure and determine whether the cough is sufficiently forceful. In yet a further alternative, the data monitoring device may also obtain secondary data to determine whether the cough was sufficiently forceful (e.g. chest expansion on the intake breath prior to the cough). In still another alternative, the clinician may decide to obtain multiple cough profiles at each measurement point (e.g. both a weak cough profile and a strong cough profile). Finally, the clinician (or the data monitoring device) may determine that a particular shape of cough profile is required. For example, a sharp cough may take less time than a deep cough.

After being instructed to continue with the test, data monitoring device 18 activates the motor for a fixed time period to pull the catheter to the second measurement point. The patient is then instructed to cough, and the clinician again determines if the cough was sufficiently forceful, as described above. The test continues until sufficiently forceful cough have been used at each data point. The clinician then completely withdraws the catheter.

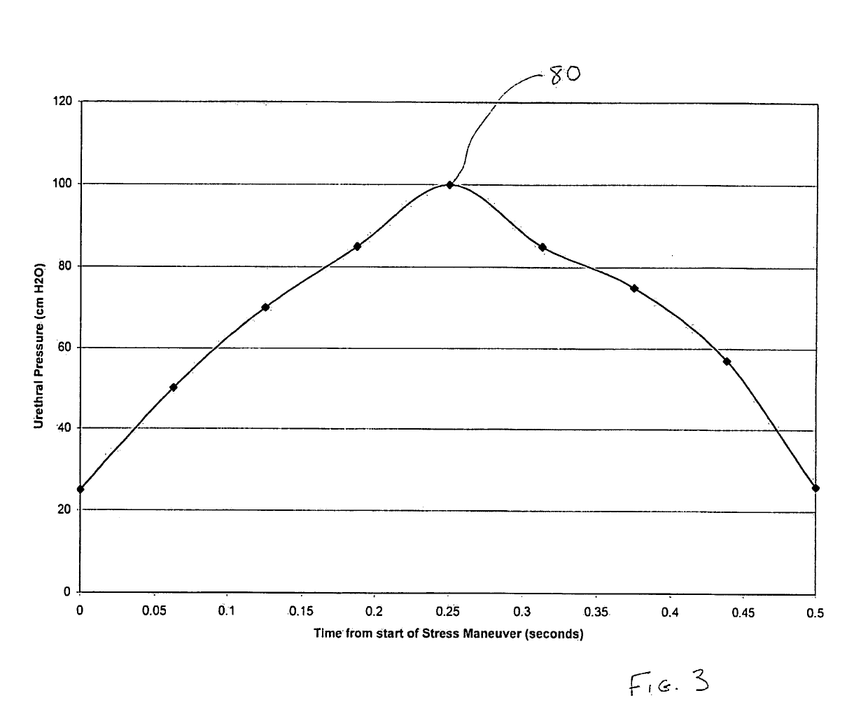

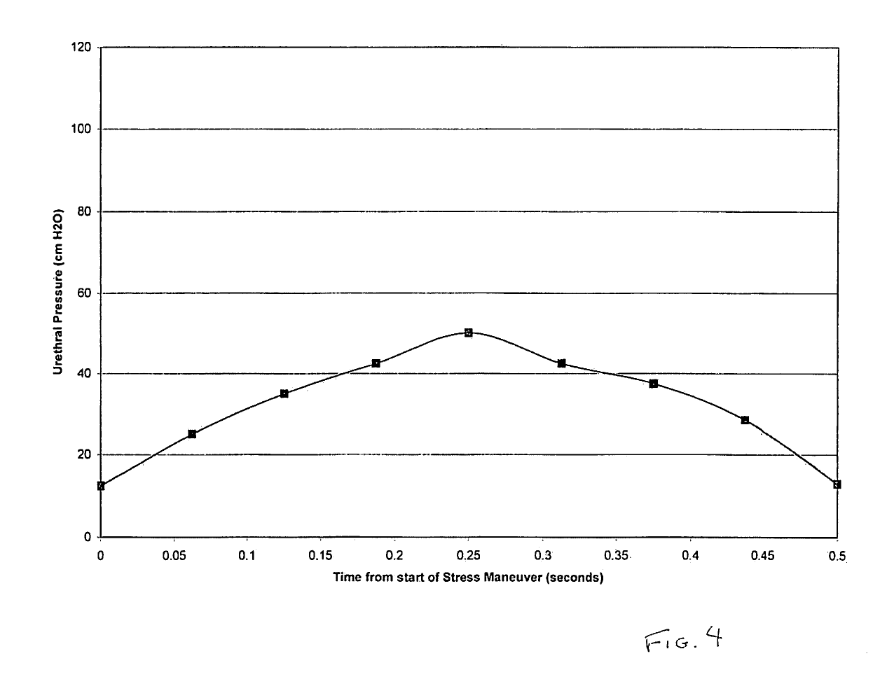

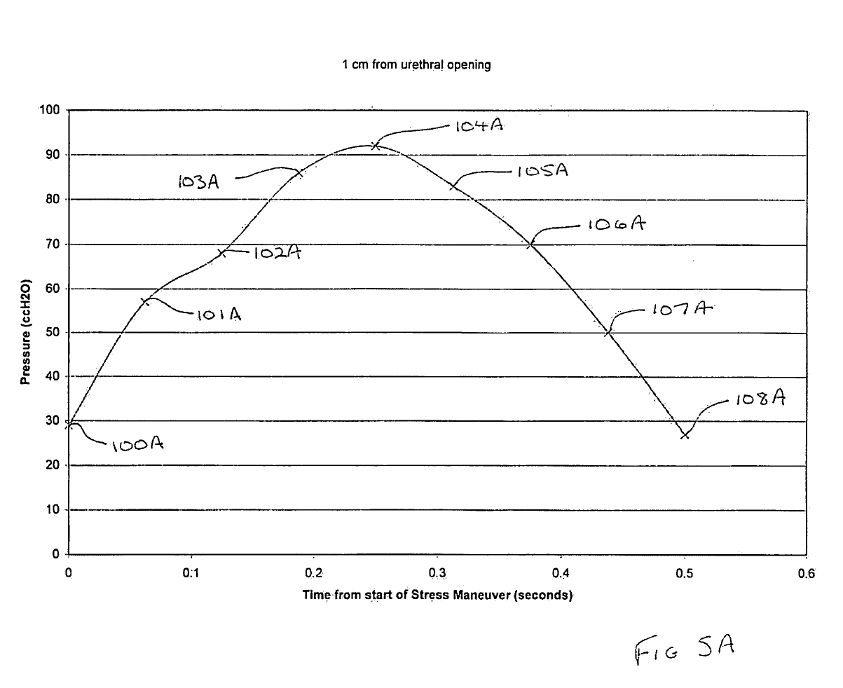

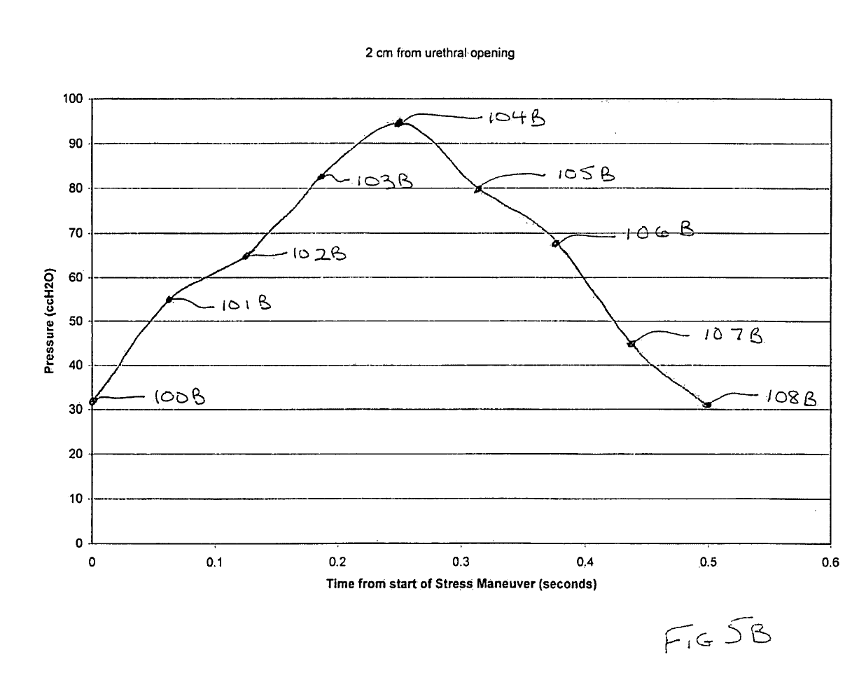

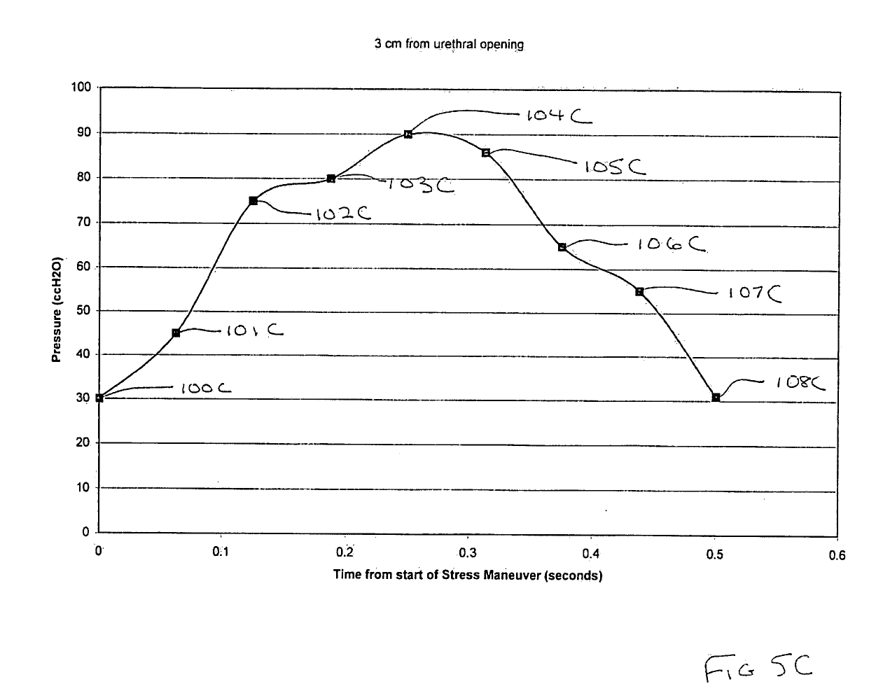

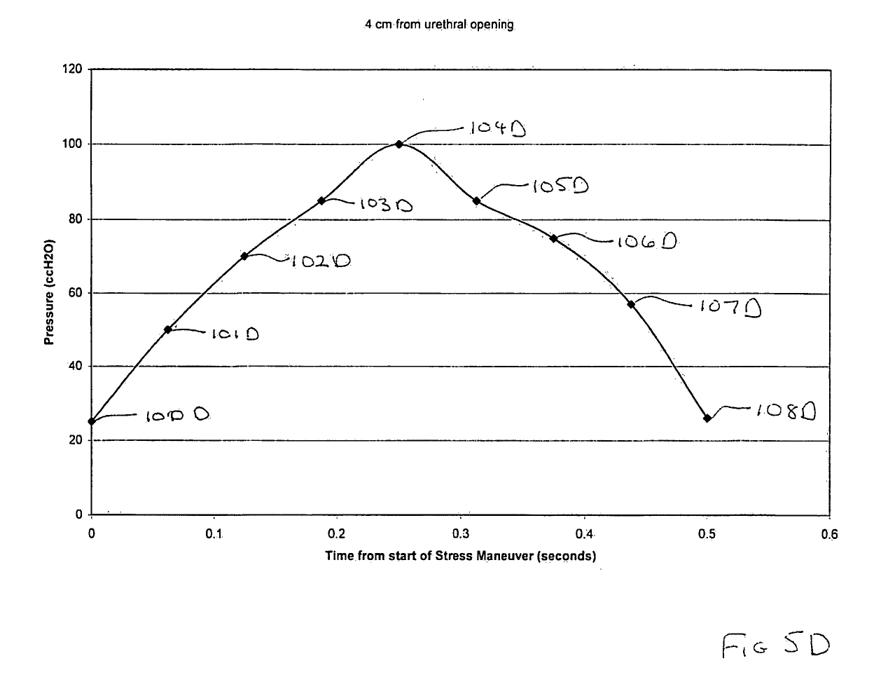

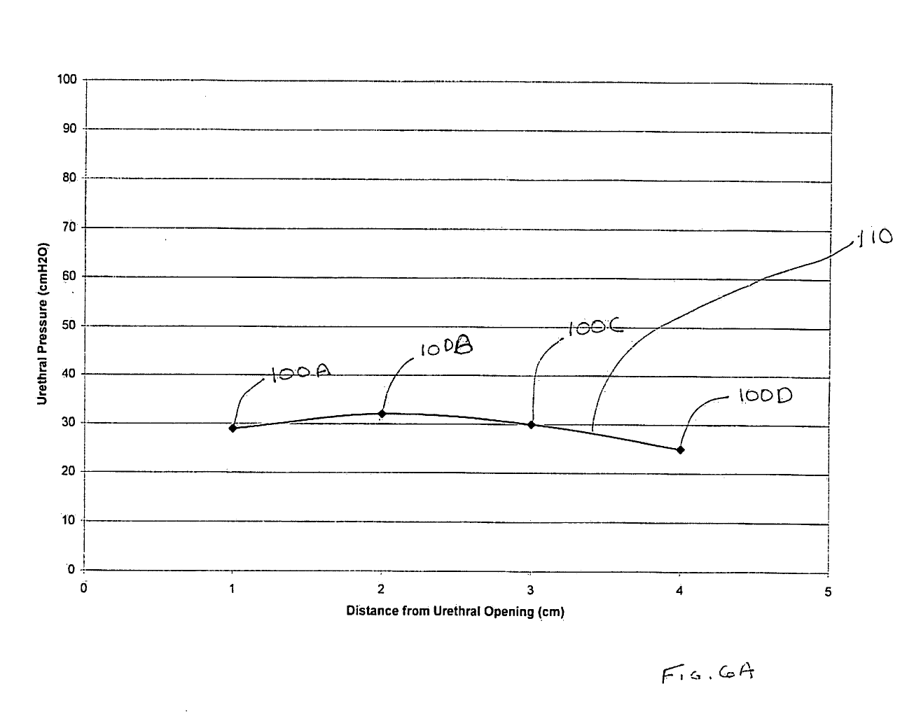

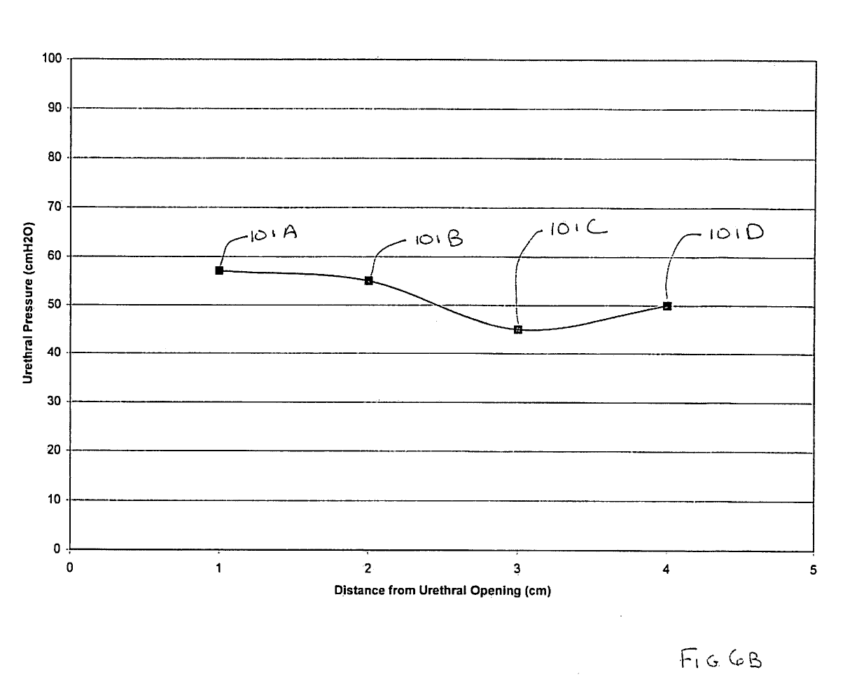

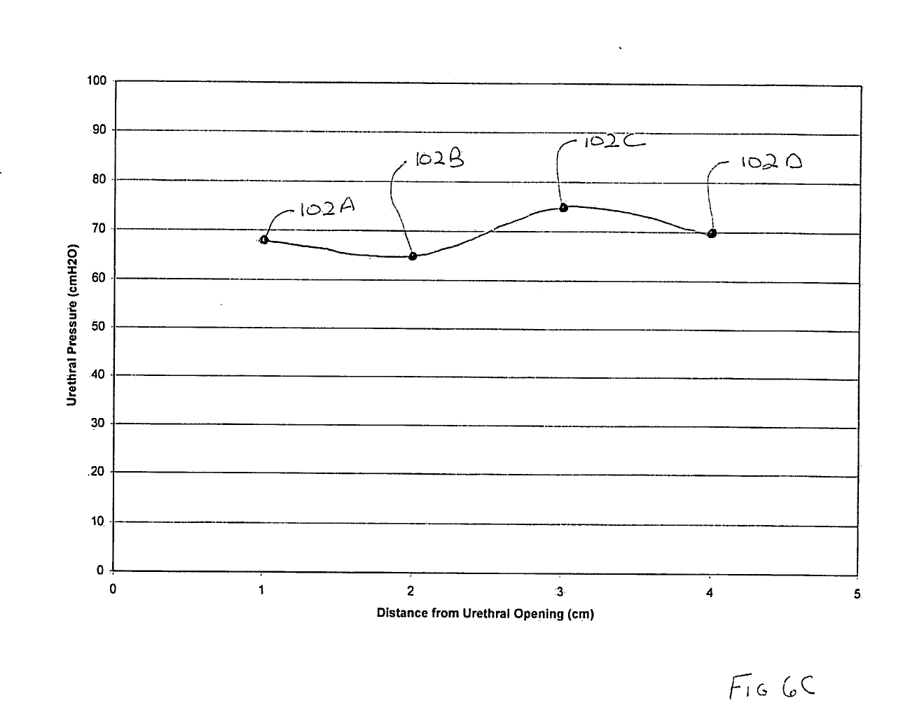

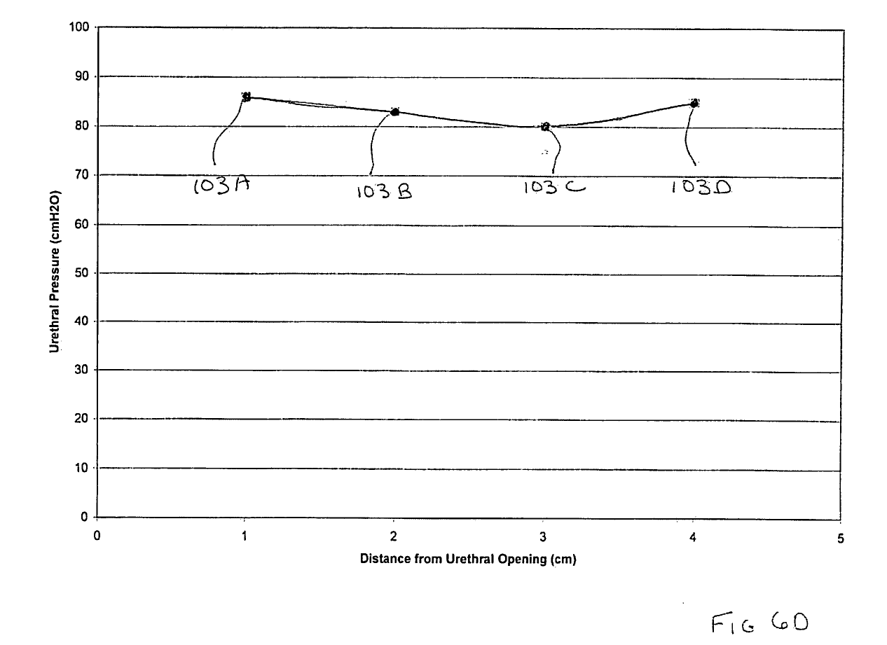

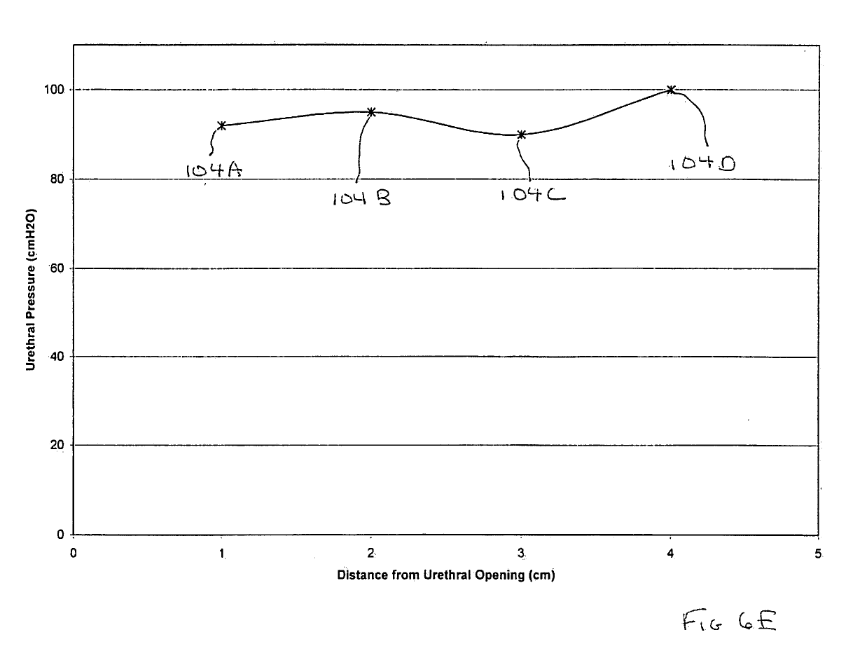

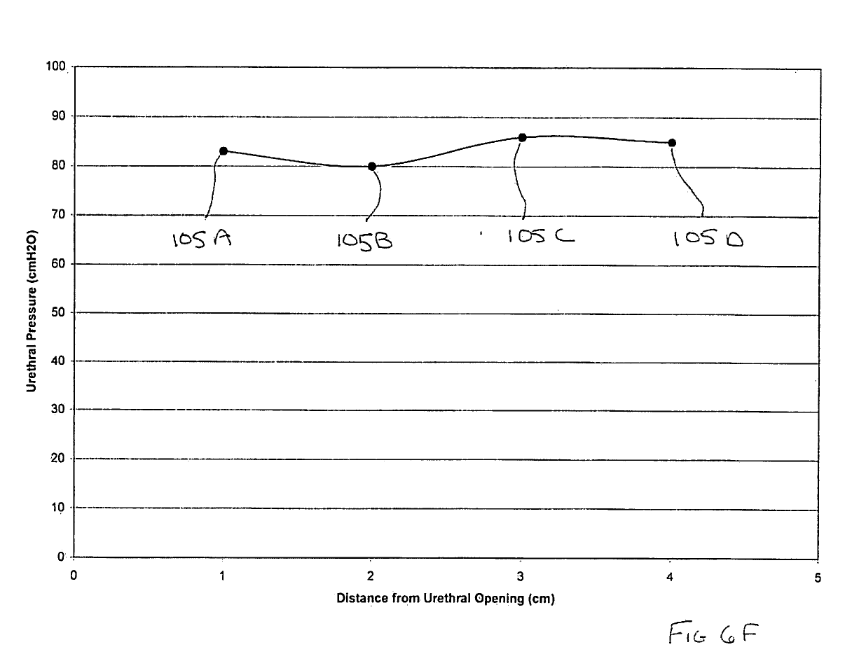

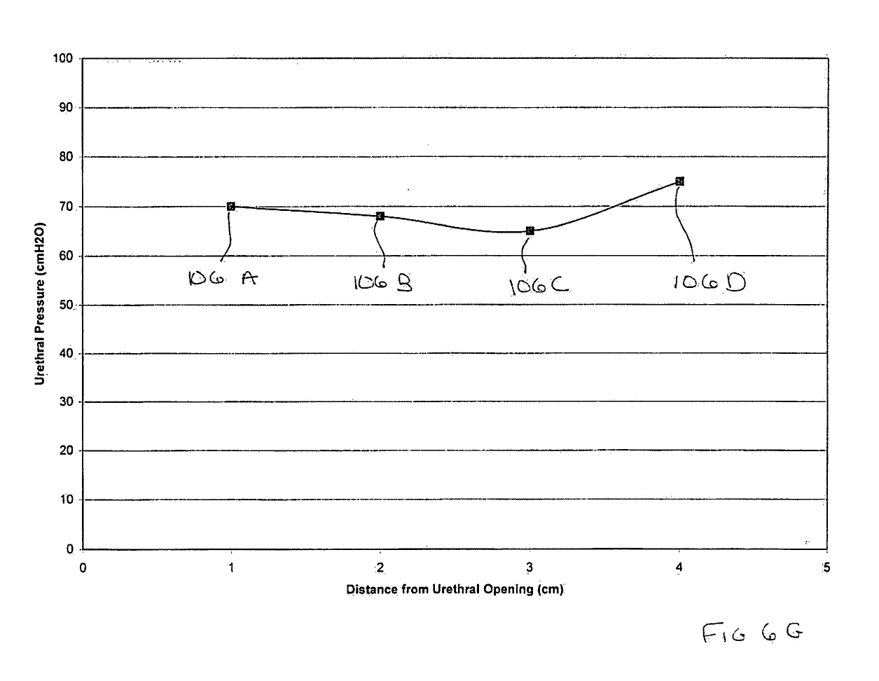

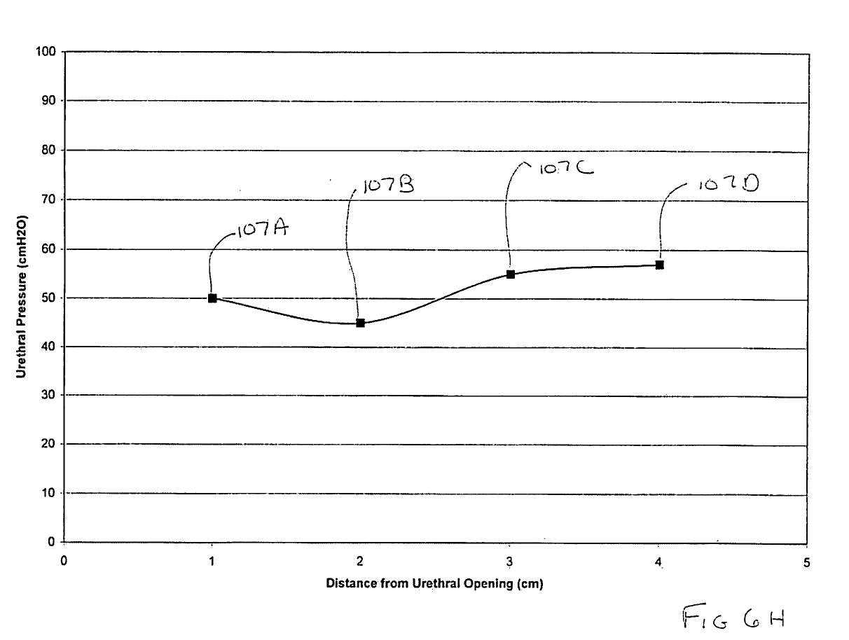

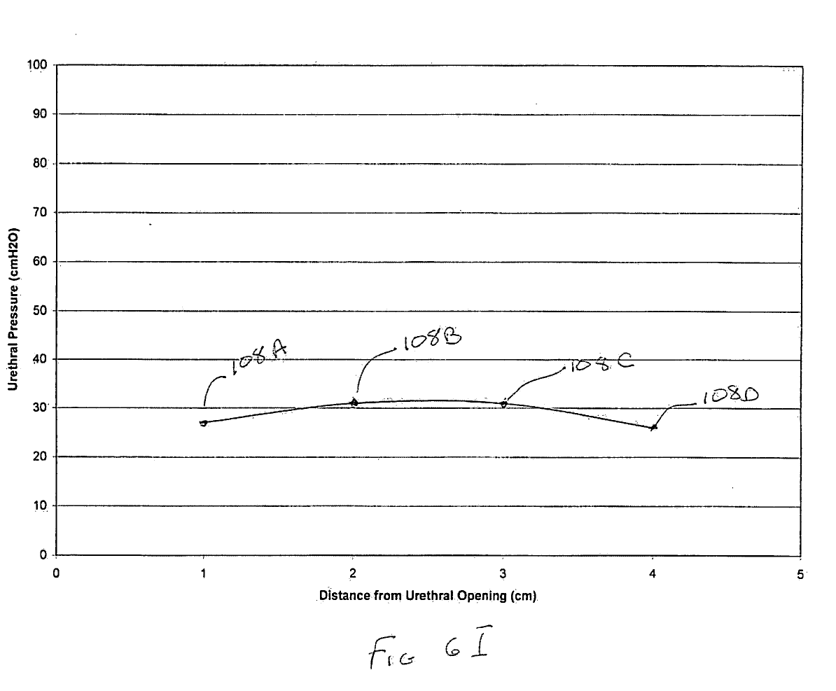

At this point, the clinician will have selected cough profiles for each measurement point along the urethra. FIGS. 5A through 5D each show a sample cough profile taken at different measurement points. The start points (data points 100A through 100D in FIGS. 5A through 5D) of each cough profile are determined. The start points may be determined in a number of ways. The clinician can enter the start time in data monitoring device 18 using a keypad connected thereto. If data monitoring device 18 includes a touch-sensitive screen, the clinician can place a mark directly on the cough profile. Alternatively, a mouse, keyboard or other input device may be used to mark the start of the cough profile. Data monitoring device 18 may be configured to interpret that mark as the starting point. Alternatively, the data monitoring device 18 may make the determination automatically based on predetermined algorithms concerning the slope of the cough profile.

At this stage, the clinician will select (or it may be pre-selected according to a template) the number of data points along each cough profile to be used for visualizing the cough profiles. In FIGS. 5A through 5D, nine data points 100 through 108 (marked as 100A through 108A on FIG. 5A, 100B through 108D on FIG. 5B etc.) are selected at fixed time intervals. Preferably, the number of data points and the time interval between them are selected such that the first data point occurs at or prior to the start of the cough, one data point is selected at or near the peak of the cough profile (i.e. maximal urethral wall pressure) and one data point is selected towards the end of the cough profile.

Each urethral pressure data point 100 through 108 may be plotted on a traditional urethral pressure profile i.e. a pressure—length profile. Examples of these urethral pressure profiles are shown in FIGS. 6A through 6I. FIG. 6A shows the data points 100A through 100D plotted on the urethral pressure profile at lengths corresponding to their respective measurement points. FIG. 6A is similar in shape to a standard unstressed urethral pressure profile as the pressure measurements are taken prior to the start of the cough. The data points in FIG. 6A are joined by a curve 110 to form the profile. Curve 110 may be determined using standard curve fitting techniques. Alternatively, as the data monitoring device 18 recorded the urethral wall pressures between the measurement points as the catheter was drawn through the urethra, this recorded data may instead be used to create the curve 110.

Subsequent data points 101A through 101D, 102A through 102D etc. are then plotted on subsequent pressure profiles, as shown in FIGS. 6B through 6I. Thus each profile represents the urethral wall pressures at various intervals during a cough. The data points for FIGS. 6B through 6I are joined by a standard curve fit as is known in the art to allow for easier visualizations. Alternatively, the data points actually recorded while the catheter was pulled through the urethra may instead be used to join the measurement points. In such a case, the urethral pressure profile will appear to be a standard, unstressed profile punctuated by four pressure spikes at each measurement point.

An alternative manner of determining data points 100 through 108 may also be used. In this alternative, the data points 100 and 108 are selected by the clinician in the normal manner (at the start and end of the cough, respectively). The clinician further selects point 104 at a time when the peak pressure is measured in each cough profile. As coughs are variable events, the peak pressure will occur at different times relative to the start of a cough. For example, the peak pressure may occur at 0.25 s from the start of one cough and at 0.35 s from the start of another cough. The remaining points (101, 102, 103, 105, 106 and 107) could be selected using a number of other methods. One method would involve dividing the time between the start of the cough (data point 100) and the time representing peak pressure (data point 104) and dividing that time into the desired number of equally spaced intervals. The data points at those intervals would then be used for plotting the urethral pressure profiles of FIGS. 6B through 6D. (Similarly, data points 105, 106 and 107 could then also be calculated for the downward slope of the cough profile.) Alternatively, data points 101, 102 and 103 may be determined at multiples of 0.25, 0.5, and 0.75, respectively, of the pressure differential between data points 100 and 104. While the resulting series of images would not necessarily be a true time-stepping visualization, they may be more useful from a clinical perspective for qualitative determinations. One possible manner in which such a visualization method may be useful is that FIG. 6E would show the peak pressure of a cough along the urethra in one image. If the duration of the patient's coughs vary throughout the test, the peak pressure for the locations may be shown in different images.

After the urethral pressure profiles are developed at each desired time interval, data monitoring device 18 may join the profiles in sequence to form a moving image. The curves may be color-coded to show areas of higher and lower pressure. Optionally, the areas under the curves may be color-coded. In this manner, the clinician can view the sequence and quickly determine whether there are any areas with lower than expected urethral wall pressures during the stressor event. The sequence may be displayed in real-time or at a slower rate. This sequence, when displayed in this manner, will form an animation from which the clinician may make diagnoses.

The clinician may view the animation to assist in determining whether the urinary sphincter is giving out under stress. In such a situation, the animated profiles may show a lower pressure in positions between the sphincter and the opening of the urethra than in positions between the sphincter and the bladder. If the animated profiles do not show this pressure pattern, the clinician may diagnose the patient with urinary leakage under stress as having a neurogenic disorder where the sphincter relaxes under stress instead of closing.

A person skilled in the art can readily determine that there are a wide variety of variations possible to the present invention. If the data monitoring device 18 is an analog printer, the data points 100 through 108 will need to be determined manually and plotted manually or using graphing software.

The data monitoring device 18 may include a plurality of processing units e.g. a handheld computer for controlling the motor and prompting the clinician and a separate computer for processing the data and preparing the video.

In one variation, the points along the cough profile beyond the peak pressure may be discarded with the animation starting at the start of the cough and ending at the peak of the cough profile. In another variation, the cough profile may be assumed to be symmetrical with the measured data points between the start of the cough and the peak of the cough used to create the remainder of the cough profile beyond the peak pressure.

In another variation, the cough profiles may be normalized to one of the cough profiles. In this manner, the data of one slightly weaker cough profile (which might otherwise result in a misdiagnosis) are normalized to another profile and allows for proper diagnosis.

In another variation, the positions at which stress maneuvers are performed may be dictated by a significant change in urethral pressure. When such a pressure change is detected, the data monitor may automatically shut down the motor and indicate that a stress maneuver is required at the given position. These positions at which the pressure change occurs may be used in addition to the predetermined positions.

In another variation, if the clinician observes urinary leakage during one or more of the cough profiles, the clinician can associate the profiles or the data points with the leakage in the data monitoring device. The data monitoring device may then display the data points in the final animation in a different manner (such as a different color). This would allow the clinician to view the position and pressures at which leakage occurred.

In another variation, the sampling rate used to obtain pressure measurements could vary throughout the pulling process. For example, if the pressure between two successive measurements increases by a predetermined interval, the sampling rate could be increased to obtain greater resolution of the localized pressure difference.

In another variation, the clinician inserts the catheter 20 such that the balloon is initially inside the bladder 30 as shown in FIG. 1. The clinician can initially measure the pressure within the bladder. If the bladder pressure is not sufficient to allow for leakage, the clinician may infuse the bladder with fluid. Such an infusion could be made through a second lumen within the catheter in fluid communication with an opening in the catheter that is positionable within the bladder. The bladder pressure can be recorded prior to the test. The data monitoring device can compare the bladder pressure with the pressure recorded in the urethra at any position or time and obtain a Pressure Transmission Ratio (PTR). The PTR can be used to in the urethral pressure profiles instead of the measured pressures. The PTR can also be calculated with respect to the peak pressures recorded in a cough profile.

Optionally, the catheter 20 may have a plurality of pressure sensors mounted thereon to record pressure simultaneously at different points along the urethra. Another option is to mount a plurality of pressure sensors on the catheter 20 in a radial manner about the catheter.

In addition, clinicians may use a wide variety of stress events to form the cough profiles. While a cough is a commonly used stress maneuver, the clinician can ask the patient to perform a Valsalva maneuver in which the patient attempts to breathe outwardly while keeping the nose and mouth closed. The typical duration of a Valsalva maneuver is between 4 and 8 seconds.

Optionally, the clinician can detect the change in the functional urethral length during a cough. The functional urethral length is defined as the distance over which the pressure in the urethra is greater than the pressure in the bladder. During stress maneuvers near the junction between the urethra and the bladder, the urethral length can be determined by comparing the pressure in the bladder to the urethral pressure.

Other variations of the above principles will be apparent to those who are knowledgeable in the field of the invention, and such variations are considered to be within the scope of the present invention. Other modifications and/or alterations may be used in the design and/or manufacture of the apparatus of the present invention, without departing from the spirit and scope of the accompanying claims.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word ‘substantially’ when used with an adjective or adverb is intended to enhance the scope of the particular characteristic; e.g., substantially perpendicular is intended to mean perpendicular, nearly perpendicular and/or exhibiting characteristics associated with perpendicularity.

1. A method for performing urodynamic testing on mammals, the method comprising the steps of:
(a) inserting a pressure sensor to a first position within a urethra of the mammal, said pressure sensor adapted to transmit the pressure within the urethra to a data monitoring device;
(b) measuring the pressure within the urethra of the mammal while the mammal undergoes at least one stress maneuver;
(c) selecting one of said at least one stress maneuvers as an accepted stress maneuver;
(d) selecting a first urethral pressure measured prior to the accepted stress maneuver, a maximal urethral pressure measured during the accepted stress maneuver, and an intermediate pressure measured between the first urethral pressure and the maximal urethral pressure, said intermediate pressure occurring at a time interval from the start of said stress maneuver;
(e) moving the pressure sensor to a second position within the urethra;
(f) repeating steps (b), (c), and (d) at the second position; and
(g) displaying a timewise representation of urethral pressures at the first position and the second position during the pressure events on a display derived from the urethral pressures determined in step (d).

2. A method as claimed in claim 1, wherein the pressure sensor includes a fluid-filled element.

3. A method as claimed in claim 2, wherein the fluid filled-element is a balloon.

4. A method as claimed in claim 1, wherein the pressure sensor includes an electronic microtip.

5. A method as claimed in claim 1, wherein the pressure sensor includes an open perfused microtip.

6. A method as claimed in claim 1, wherein the timewise representation of urethral pressures is a series of graphs displaying urethral pressure as a function of urethral location.

7. A method as claimed in claim 6, wherein one of said graphs displays the first urethral pressure at said first position and said second position.

8. A method as claimed in claim 7, wherein one of said graphs displays the maximal urethral pressure at said first position and said second position.

9. A method as claimed in claim 6, wherein one of said graphs displays the intermediate urethral pressure at said first position and said second position.

10. A method as claimed in claim 1, wherein steps (b), (c) and (d) are repeated at a third position.

11. A method as claimed in claim 10, wherein the timewise representation of urethral pressures is a series of graphs displaying urethral pressure as a function of urethral location.

12. A method as claimed in claim 1 1, wherein one of said graphs displays a pressure at said first position, said second position and said third position selected from the group consisting of: the first urethral pressure, the maximal urethral pressure and the intermediate pressure.

13. A method as claimed in claim 12, wherein said pressures are displayed as points on said graph.

14. A method as claimed in claim 13, wherein said points are joined using a curve-fitting algorithm.

15. A method as claimed in claim 1, wherein the pressure in the bladder of the mammal is also measured.

16. A method as claimed in claim 1, wherein the stress maneuver is a cough performed by the mammal.

17. A method as claimed in claim 1, wherein the stress maneuver is a Valsalva maneuver performed by the mammal.

18. A method as claimed in claim 1, wherein step (e) is performed using a motorized puller.

19. A method as claimed in claim 1, wherein said mammal is observed for indications of urinary leakage.

20. A method as claimed in claim 1, wherein the pressure in the bladder of the mammal is also measured and wherein each of said measured urethral pressures is expressed as a percentage of bladder pressure.

21. A method as claimed in claim 1, wherein a stress profile for each of said first position and said second position is prepared, said stress profile displaying said first urethral pressure, said maximal urethral pressure and said intermediate urethral pressure as a function of time.

22. A method as claimed in claim 21, wherein said stress profiles are normalized with respect to said bladder pressure.

23. A method as claimed in claim 1, wherein said maximal urethral pressure is selected by selecting the pressure measured at a preselected time interval from the start of the stress maneuver.

24. A method as claimed in claim 1, wherein said intermediate urethral pressure is selected by selecting the pressure measured at a preselected time interval from the start of the stress maneuver.

25. A method as claimed in claim 1, wherein said first position and said second position are selected at predetermined distances from the opening of the urethra.

26. A method for displaying urethral pressure profiles for mammals comprising the steps of:
(a) obtaining pressure measurements at a plurality of locations within the urethra of a mammal while the mammal undergoes a stress maneuver, said pressure measurements being plottable on a pressure-time graph as a stress profile;
(b) selecting a first pressure measurement obtained in step (a) from each location within the urethra used in step (a), said first pressure measurements selected such that they occur at substantially corresponding points in the respective stress profiles, said first pressure measurements forming a first set of profile pressures;
(c) selecting a second pressure measurement obtained in step (a) from each location within the urethra used in step (a), said second pressure measurements selected such that they occur at substantially corresponding points in the respective stress profiles, said second pressure measurements forming a second set of profile pressures;
(d) plotting each of said first pressure measurements on a first graph of pressure as a function of urethral location;
(e) plotting each of said second pressure measurements on a second graph of pressure as a function of urethral locations; and
(f) displaying each of said graphs in sequential manner.

27. A method as claimed in claim 26, wherein the first pressure measurements are joined together on a curve.

28. A method as claimed in claim 27, wherein the curve is calculated using a curve-fitting algorithm.

29. A method as claimed in claim 26, wherein the stress profiles are normalized with respect to a preselected pressure.

30. A method as claimed in claim 26, wherein said graphs are displayed on a computer display.
